(19) United States
(12) Patent Application Publication
(10) Pub. No.: US 2021/0165488 A1
(43) Pub. Date: Jun. 3, 2021

(54) INFORMATION PROCESSING APPARATUS AND NON-TRANSITORY COMPUTER READABLE MEDIUM

(71) Applicant: FUJI XEROX CO., LTD., Tokyo (JP)

(72) Inventors: Kengo TOKUCHI, Kanagawa (JP); Masahiro SATO, Kanagawa (JP)

(73) Assignee: FUJI XEROX CO., LTD., Tokyo (JP)

(21) Appl. No.: 16/857,752

(22) Filed: Apr. 24, 2020

(30) Foreign Application Priority Data
Dec. 3, 2019 (JP) 2019-219117

(51) Int. Cl. G06F 3/01; G05B 15/02

(52) U.S. Cl. CPC G06F 3/015; G05B 15/02; G06F 3/017

(57) ABSTRACT

An information processing apparatus includes first and second sensors and a processor. The first sensor monitors specific biological information concerning a user. The specific biological information is used for operating a device. The second sensor identifies a part of the user where the first sensor is placed. The processor is configured to operate the device in accordance with the specific biological information monitored by the first sensor when the second sensor has determined that the first sensor is placed at a specific part where the specific biological information is monitored.

### CROSS-REFERENCE TO RELATED APPLICATIONS

This application is based on and claims priority under 35 USC 119 from Japanese Patent Application No. 2019-219117 filed Dec. 3, 2019.

### BACKGROUND

### (i) Technical Field

The present disclosure relates to an information processing apparatus and a non-transitory computer readable medium.

### (ii) Related Art

A device may be operated by the use of biological information, such as brain waves.

Japanese Unexamined Patent Application Publication No. H10-099287 discloses a method for correcting the cerebral evoked potential, based on the value obtained by measuring skin impedance.

### SUMMARY

A sensor, which monitors specific biological information for operating a device, is placed at a specific part of a user where this specific biological information is monitored. However, it is still possible that the sensor be placed at a different part from the specific part. Even if the sensor is placed at a different part, biological information can still be monitored, and the device may unfavorably be operated based on this biological information.

Aspects of non-limiting embodiments of the present disclosure relate to a structure that makes it possible to operate a device when a sensor, which monitors specific biological information for operating the device, is placed at a specific part where the specific biological information is monitored.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided an information processing apparatus including first and second sensors and a processor. The first sensor monitors specific biological information concerning a user. The specific biological information is used for operating a device. The second sensor identifies a part of the user where the first sensor is placed. The processor is configured to operate the device in accordance with the specific biological information monitored by the first sensor when the second sensor has determined that the first sensor is placed at a specific part where the specific biological information is monitored.

### BRIEF DESCRIPTION OF THE DRAWINGS

An exemplary embodiment of the present disclosure will be described in detail based on the following figures, wherein:

FIG. 1 is a block diagram illustrating an example of the configuration of an information processing system according to the exemplary embodiment;

FIG. 2 is a block diagram illustrating an example of the configuration of an information processing apparatus according to the exemplary embodiment;

FIG. 3 illustrates a management table;

FIG. 4 illustrates another management table; and

FIG. 5 illustrates a schematic model of a human body.

### DETAILED DESCRIPTION

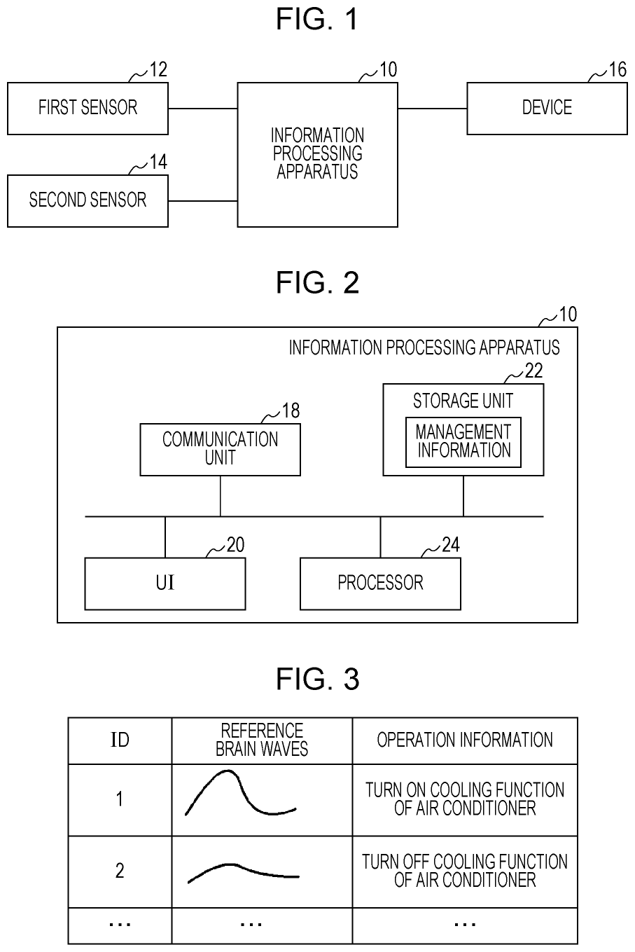

An information processing system according to the exemplary embodiment will be described below with reference to FIG. 1. FIG. 1 illustrates an example of the configuration of the information processing system according to the exemplary embodiment.

The information processing system according to the exemplary embodiment includes an information processing apparatus 10, one or plural first sensors 12, one or plural second sensors 14, and one or plural devices 16. The single information processing apparatus 10, first sensor 12, second sensor 14, and device 16 are shown in FIG. 1, but this configuration is only an example. Plural first sensors 12, plural second sensors 14, and plural devices 16 may be included in the information processing system. The information processing system may include a device (an external device, such as a server, for example) other than the information processing apparatus 10, the first and second sensors 12 and 14, and the device 16 shown in FIG. 1.

Each apparatus or device (information processing apparatus 10, first and second sensors 12 and 14, and device 16, for example) included in the information processing system is configured to communicate with another apparatus or device included in the information processing system via a wired medium using a cable or a wireless medium. That is, each apparatus or device may be physically connected to another apparatus or device via a cable and send and receive information with each other. Alternatively, each apparatus or device may send and receive information with another apparatus or device by wireless communication. Examples of wireless communication are short-range wireless communication and Wi-Fi (registered trademark). Another wireless communication standard may be employed. Examples of short-range wireless communication are Bluetooth (registered trademark), radio frequency identifier (RFID), and near field communication (NFC). Each apparatus or device may alternatively communicate with another apparatus or device via a communication channel, such as a local area network (LAN) or the Internet.

Examples of the information processing apparatus 10 are a personal computer (hereinafter called a PC), a tablet PC, a smartphone, a cellular phone, and other apparatuses, such as a server. The information processing apparatus 10 may be a portable terminal device (such as a tablet PC, a smartphone, or a cellular phone), or a device placed on a desk or a table. The information processing apparatus 10 may be a smart speaker having a communication function and including a microphone and a speaker. The information processing apparatus 10 may be installed indoors (on the floor or the ceiling of a room or on the table in a room) or outdoors. The information processing apparatus 10 may be a movable device, such as a self-running device.

The first sensor 12 is a sensor that monitors biological information concerning a user, and includes electrodes, for example. The first sensor 12 may be a biological information monitoring apparatus that monitors biological information. If multiple first sensors 12 are used, they may individually monitor different types of biological information. Alternatively, some or all of the first sensors 12 may monitor the same type of biological information. Conversely, the single first sensor 12 may monitor one type of biological information or multiple types of biological information.

The first sensor 12 sends monitored biological information to the information processing apparatus 10. Every time the first sensor 12 monitors biological information, it may send the monitored information to the information processing apparatus 10. The first sensor 12 may alternatively store monitored biological information and send it to the information processing apparatus 10 at predetermined regular intervals or at a timing given by a user. The first sensor 12 may receive biological information monitored by another first sensor 12 and send it together with biological information monitored by the first sensor 12 to the information processing apparatus 10.

The first sensor 12 may analyze biological information monitored by the first sensor 12 or another first sensor 12 and send information indicating the analysis results to the information processing apparatus 10. The first sensor 12 may include a processor and a storage unit, and the processor may analyze biological information. Biological information may alternatively be analyzed by the information processing apparatus 10.

The first sensor 12 may include batteries and be driven by power supplied from them. The first sensor 12 may alternatively be driven by receiving power from the information processing apparatus 10.

The first sensor 12 may be attached to a user. For example, the first sensor 12 may be a wearable device that is worn by a user and monitors biological information concerning the user. Specific examples of the first sensor 12 are a device fixed on the user's head (the forehead, for example), a bearable device attached to the user's ear/ears (such as earphone/earphones or headphones), a device fixed to the user's arm, hand, wrist, or finger (for example, a watch-type device, such as a smart watch), a device put on the user's neck, a device fixed to the user's torso (the abdomen or chest, for example), and a device fixed to the lower limb (the thigh, lower leg, knee, foot, or ankle, for example). The first sensor 12 may be health equipment fixed to the user's arm, hand, torso, or lower limb. The first sensor 12 may be attached to another part of the body other than the above-described parts. The first sensor 12 may be attached to each of multiple parts of the body.

Biological information is physiological information and anatomical information concerning various physiological and anatomical aspects of human bodies. The concept of biological information covers information concerning the brain activities (such as brain waves (for example, electroencephalogram (EEG) monitored by electroencephalography (EEG)), brain blood flow, and brain magnetic field signal), the pulse rate, the blood pressure, the blood flow, the heart rate, the electrocardiogram waveforms, myoelectric waveforms, the eye movement, the body temperature, the amount of perspiration, gaze, voice, the amount of saliva, and the movement of a user. The above-described items of information are only examples of the biological information, and another item of physiological information or anatomical information may be used as the biological information. The first sensor 12 may monitor one or multiple items of the above-described items of biological information.

The concept of biological information covers bioelectric potential information indicating the potentials generated from the body. The concept of bioelectric potential information covers brain waves obtained by measuring minute electric currents generated by the brain activities, electrocardiograms created by measuring minute electric currents generated by the heart pulsating beats, electromyograms created by measuring minute electric currents generated by the muscle activities, and skin potentials obtained by measuring minute electric currents generated in the skin. The above-described items of information are only examples of bioelectric potential information, and another item of bioelectric potential information may be used.

After receiving biological information from the first sensor 12, the information processing apparatus 10 analyzes, stores, and outputs the biological information, and also stores and outputs information indicating the analysis results of the biological information. Analyzing of biological information may alternatively be conducted by the first sensor 12 or another device, such as a server. To output biological information is to display it or output it as sound information, for example. To output information indicating the analysis results of biological information is to display it or output it as sound information, for example. The information processing apparatus 10 may send biological information and information indicating the analysis results to another apparatus.

The information processing apparatus 10 may contain one or plural first sensors 12. That is, one or plural first sensors 12 may be integrated into the information processing apparatus 10 so as to form a single apparatus. The entirety of the information processing apparatus 10 integrating one or plural first sensors 12 may be worn by a user to monitor biological information. That is, the information processing apparatus 10 may be a wearable device. Specific examples of the information processing apparatus 10 are a device fixed on the user's head (the forehead, for example), a bearable device attached to the user's ear/ears (such as earphone/earphones or headphones), a device fixed to the user's arm, hand, wrist, or finger (for example, a watch-type device, such as a smart watch), a device put on the user's neck, a device fixed to the user's torso (the abdomen or chest, for example), and a device fixed to the lower limb (the thigh, lower leg, knee, foot, or ankle, for example). The information processing apparatus 10 may be health equipment fixed to the user's hand, torso, or lower limb. The information processing apparatus 10 may be attached to another part of the body other than the above-described parts.

The information processing apparatus 10 and the first sensor 12 may be separate apparatuses. For example, the information processing apparatus 10 may be a smart speaker, while the first sensor 12 may be a wearable device worn by a user.

The second sensor 14 is a sensor that identifies a part of a user where the first sensor 12 is placed. More specifically, the second sensor 14 is a sensor that measures data for identifying a part of a user where the first sensor 12 is placed. Examples of the second sensor 14 are a temperature sensor that measures the temperature, a motion sensor that measures the amount of movement, such as a gyroscope and an acceleration sensor, an illuminance sensor that measures the illuminance level, an odor sensor that measures the odor level, an image capturing device, such as a camera, a pressure sensor that measures the pressure level, and a humidity sensor that measures the humidity level. Another type of sensor other than the above-described types may be used as the second sensor 14. One or multiple types of sensors among the above-described types may each be included in the information processing system as the second sensor 14.

The second sensor 14 sends measured data to the information processing apparatus 10. Every time the second sensor 14 measures data, it may send it to the information processing apparatus 10. The second sensor 14 may alternatively store measured data and send it to the information processing apparatus 10 at predetermined regular intervals or at a timing given by a user. The second sensor 14 may receive data measured by another second sensor 14 and send it together with data measured by the second sensor 14 to the information processing apparatus 10.

The second sensor 14 may analyze data measured by the second sensor 14 or another second sensor 14 and send information indicating the analysis results to the information processing apparatus 10. The second sensor 14 may include a processor and a storage unit, and the processor may analyze data. Data may alternatively be analyzed by the information processing apparatus 10.

The second sensor 14 may include batteries and be driven by power supplied from the batteries. The second sensor 14 may alternatively be driven by receiving power from the information processing apparatus 10.

The second sensor 14 may be attached to the first sensor 12 or be located separately from the first sensor 12. The second sensor 14 may be attached to a user. For example, the second sensor 14 may be a wearable device worn by a user.

The information processing apparatus 10 may contain one or plural second sensors 14. That is, one or plural second sensors 14 may be integrated into the information processing apparatus 10 so as to form a single apparatus. As in the information processing apparatus 10 integrating one or plural first sensors 12, the entirety of the information processing apparatus 10 integrating one or plural second sensors 14 may be worn by a user. The information processing apparatus 10 and the second sensor 14 may be separate apparatuses.

The information processing apparatus 10 may contain one or plural first sensors 12 and one or plural second sensors 14. That is, one or plural first sensors 12 and one or plural second sensors 14 may be integrated into the information processing apparatus 10 so as to form a single apparatus. The entirety of the information processing apparatus 10 integrating one or plural first sensors 12 and one or plural second sensors 14 may be worn by a user.

Examples of the device 16 are a PC, a tablet PC, a smartphone, a cellular phone, a robot (such as a humanoid robot, an animal robot, a robotic vacuum cleaner, and another type of robot), a projector, a display, such as a liquid crystal display, a recorder, a playback device, an image capturing device, such as a camera, a refrigerator, a rice steamer, a microwave oven, a coffee machine, a vacuum cleaner, a washing machine, an air conditioner, lighting equipment, health equipment, a watch, a clock, a surveillance camera, an automobile, a motorbike, a motorcycle, a bicycle, an aircraft (an unmanned aerial vehicle, such as a drone, for example), a game machine, a gas stove, an electronic bidet, a ventilation fan, a doorbell, an entrance monitoring system, an elevator, a door, a window, and various types of sensing devices (such as a temperature sensor, a humidity sensor, a voltage sensor, and a current sensor). The concept of the device 16 may cover almost all types of equipment. For example, information equipment, video equipment, and audio equipment may be examples of the device 16 in the exemplary embodiment.

The device 16 includes a communication unit, which is a communication interface, a storage unit that stores data, and a processor that controls the operation of the device 16. The device 16 may also include a user interface. The device 16 may send device identification information for identifying the device 16 to the information processing apparatus 10. The device identification information is the ID, name, model type, or address (such as media access control (MAC) address or Internet protocol (IP) address) of the device 16, for example.

The information processing system including the information processing apparatus 10, the first and second sensors 12 and 14, and the device 16 may serve as a single apparatus. For example, the information processing apparatus 10, the first and second sensors 12 and 14, and the device 16 form one apparatus, and the single apparatus may be worn by a user. For example, the information processing apparatus 10, the first and second sensors 12 and 14, and the device 16 may form health equipment, and the health equipment may be attached to the arm, hand, torso, or lower limb of a user. For example, the health equipment may be a device that vibrates to help a user strengthen the muscles or consume more calories. The health equipment is only an example, and another type of apparatus may be formed.

The configuration of the information processing apparatus 10 will be described below in detail with reference to FIG. 2. FIG. 2 illustrates an example of the configuration of the information processing apparatus 10.

The information processing apparatus 10 includes a communication unit 18, a user interface (UI) 20, a storage unit 22, and a processor 24. The information processing apparatus 10 may include another element other than the above-described elements.

The communication unit 18, which is a communication interface, has a function of sending data to another apparatus and a function of receiving data from another apparatus. The communication unit 18 may have a wireless communication function or a wired communication function. The communication unit 18 may communicate with another apparatus by using short-range wireless communication or via a communication channel, such as a LAN or the Internet. For example, the communication unit 18 receives biological information sent from the first sensor 12 and data sent from the second sensor 14. The communication unit 18 may send control information for controlling the operation of the first sensor 12 to the first sensor 12 and control information for controlling the operation of the second sensor 14 to the second sensor 14. The communication unit 18 sends control information for controlling the operation of the device 16 to the device 16. The communication unit 18 may receive information sent from the device 16.

The UI 20 includes at least one of a display and an operation unit. The display is a liquid crystal display or an electroluminescence (EL) display, for example. The operation unit is a keyboard, input keys, or an operation panel, for example. The UI 20 may be a touchscreen which serves both as the display and the operation unit. The UI 20 may include a microphone and/or a speaker which emits sound.

The storage unit 22 is a device forming one or multiple storage regions for storing data. The storage unit 22 is a hard disk drive, various memory devices (such as a random access memory (RAM), a dynamic random access memory (DRAM), and a read only memory (ROM)), another storage unit (such as an optical disc), or a combination thereof, for example. One or plural storage units 22 are included in the information processing apparatus 10.

In the storage unit 22, management information is stored. Management information is information for determining the operation content of the device 16, based on monitored biological information concerning a user. For example, predetermined reference biological information and operation information indicating the operation content of the device 16 are linked with each other and are registered in the management information. Reference biological information may be biological information that is assumed to be generated from a user who performs the operation linked with this reference biological information, or biological information that is assumed to be generated from a user who makes a request to perform this operation. It can be said that the reference biological information is biological information associated with the operation content of the device 16. For each user, reference biological information and operation information may be linked with each other and be registered in the management information.

In the management information, operation information concerning the operation content of the power supply of the device 16 or operation information concerning the operation content of a function level of the device 16 may be registered. Alternatively, these two items of operation information may be registered.

The operation content of the power supply of the device 16 is the operation for turning ON or OFF the power supply of the device 16. The operation information concerning the operation content of the power supply is information indicating the operation for turning ON or OFF the power supply of the device 16. It can be said that the biological information linked with the operation information concerning the operation content of the power supply is biological information associated with the ON/OFF operation of the power supply of the device 16. For each user, reference biological information and operation information concerning the operation content of the power supply of the device 16 may be linked with each other and be registered in the management information.

The operation content of a function level of the device 16 is the operation for setting a function level of the device 16. The operation information concerning the operation content of a function level is information indicating the operation for setting a function level of the device 16. It can be said that the biological information linked with the operation information concerning the operation content of a function level is biological information associated with the function level of the device 16. For each user, reference biological information and operation information concerning the operation content of a function level of the device 16 may be linked with each other and be registered in the management information.

The function level is a level of the performance or the output of the device 16. Examples of the function level are the set temperature, airflow volume, and airflow direction of an air conditioner, the ON/OFF state of a dehumidifying function of an air conditioner, the luminance of a display, the luminance of lighting equipment, the volume level of a speaker, the moving speed of a self-running device (such as a robot and a self-running vacuum cleaner), the set values of various devices, such as an image capturing device, a recorder, and a playback device, the set values of household appliances, such as a refrigerator, a rice steamer, and a microwave oven, and the set values of various sensing devices. These are only examples of the function level, and another level or value may be used.

The processor 24 is configured to obtain biological information concerning a user and to output an instruction to operate the device 16 in accordance with the biological information.

For example, when biological information concerning a user is monitored by the first sensor 12, it is sent from the first sensor 12 to the information processing apparatus 10. The processor 24 receives the biological information and determines the operation content of the device 16 based on the biological information. The processor 24 sends control information including the operation information indicating the determined operation content to the device 16, thereby operating the device 16. Upon receiving the control information, the device 16 is operated in accordance with this control information. Processing for determining the operation content of the device 16 based on the biological information may be executed by another apparatus, such as a server, or by the first sensor 12, instead of the information processing apparatus 10. In this case, the operation information indicating the determined operation content is sent from another apparatus or the first sensor 12 to the information processing apparatus 10, and the processor 24 receives this operation information.

For example, the processor 24 checks monitored biological information concerning a user against each item of reference biological information registered in the management information, and searches for an item of reference biological information which deviates from the monitored biological information by an amount within a permissible range. The processor 24 then identifies the operation content of the device 16 linked with this item of reference biological information. In this manner, the device 16 to be operated and the operation content of the device 16 are determined. The permissible range concerning the difference in biological information is determined in advance. The permissible range may be changed by a user. The permissible range may be set for each user.

If multiple items of reference biological information which each deviate from the monitored biological information by an amount within the permissible range are found, the processor 24 selects the item of reference biological information having the smallest difference from the monitored biological information. The processor 24 then identifies the operation content of the device 16 linked with this item of reference biological information.

The reference biological information may be information indicating feature components of biological information. In this case, the processor 24 may extract feature components from monitored biological information concerning a user, and search for an item of reference biological information whose feature components are different from those of the monitored biological information by an amount within the permissible range. For example, if brain waves are used as biological information, the processor 24 may extract feature components from monitored brain waves, analyze them, and estimate the operation content associated with the analyzed brain waves.

The first sensor 12 that monitors brain activities and the information processing apparatus 10 may form a brain-machine interface. For this brain-machine interface, either one of the invasive approach or the non-invasive approach may be employed. The processor 24 operates the device 16 based on the brain activities (brain waves, for example) of a user. To operate the device 16, the processor 24 may extract feature components from the brain waves and operate the device 16 based on the extracted feature components. To extract feature components from brain waves, fast Fourier transform (FFT), wavelet transform (WT), time frequency distribution (TFD), eigenvector methods (EM), or auto regressive method (ARM) may be used. As the approach to linking the brain waves and the operation content of the device 16 by using feature vectors obtained from the extracted feature components, independent component analysis (ICA), k-means clustering, support vector machine (SVM), or convolutional neural network (CNN) may be used.

The processor 24 may receive device identification information sent from the device 16 and identify the device 16. For example, the processor 24 sends a request to acquire device identification information to the device 16 and receives device identification information sent from the device 16. If the device 16 is connected to the information processing apparatus 10 so that they can communicate with each other, the device 16 may directly send device identification information to the information processing apparatus 10, and the processor 24 receives it.

The processor 24 is configured to control the operations of the individual elements of the information processing apparatus 10. The processor 24 may include a memory.

If the second sensor 14 has determined that the first sensor 12 is placed at a specific part of a user where specific biological information is monitored, the processor 24 is configured to operate the device 16 in accordance with the specific biological information monitored by the first sensor 12.

The specific biological information is biological information for operating a subject device 16. The specific part is a part related to this subject device 16 and is the part where the specific biological information is monitored. That is, when the first sensor 12 is placed at the specific part, the specific biological information for operating the subject device 16 is monitored by the first sensor 12. In other words, the subject device 16 is a device that is assumed to be operated in accordance with the specific biological information monitored by the first sensor 12 placed at the specific part related to the subject device 16.

Details of processing to be executed by the processor 24 will be discussed below. The processor 24 receives data measured by the second sensor 14 from the second sensor 14. Based on this data, the processor 24 identifies the part of a user where the first sensor 12 is placed.

If the part of a user where the first sensor 12 is placed is the specific part of the user where the specific biological information for operating the subject device 16 is monitored, the processor 24 operates the device 16 in accordance with the biological information monitored by the first sensor 12.

If the part of a user where the first sensor 12 is placed is the specific part, the processor 24 may notify the user that the first sensor 12 is placed at the specific part. For example, the processor 24 may display on the display of the UI 20 information indicating that the first sensor 12 is placed at the specific part or output this information from a speaker as sound information. If the user has given an instruction to operate the device 16 in accordance with biological information monitored by the first sensor 12, the processor 24 may operate the device 16 in accordance with this biological information. Even without receiving an instruction from a user, the processor 24 may operate the device 16 in accordance with biological information monitored by the first sensor 12 placed at the specific part.

If the part of a user where the first sensor 12 is placed is not the specific part where the specific biological information for operating the subject device 16 is monitored, the processor 24 does not operate the device 16 in accordance with the biological information monitored by the first sensor 12.

If the part of a user where the first sensor 12 is placed is not the specific part, the processor 24 may notify the user that the first sensor 12 is not placed at the specific part. For example, the processor 24 may display on the display of the UI 20 information indicating that the first sensor 12 is not placed at the specific part or output this information from a speaker as sound information. Even if the first sensor 12 is not placed at the specific part, the user may give an instruction to operate the device 16 in accordance with biological information monitored by the first sensor 12. In this case, the processor 24 may operate the device 16 in accordance with this biological information.

Processing to be executed by the processor 24 will be discussed below through illustration of a specific example. If the subject device 16 is a device which is assumed to be operated based on brain waves, the specific biological information is brain waves, and the specific part is a part where brain waves are monitored. The part where brain waves are monitored is the head, for example, and is more specifically the forehead or the ear (ear canal, for example).

If it is determined based on data measured by the second sensor 14 that the first sensor 12 for monitoring brain waves is placed at the specific part (the forehead or the ear, for example) where brain waves are monitored, the processor 24 operates the device 16 in accordance with biological information monitored by the first sensor 12. That is, if it is determined that the first sensor 12 is placed at the specific part where brain waves are monitored, it is likely that biological information to be monitored by the first sensor 12 is brain waves. In this case, the processor 24 operates the device 16 in accordance with the biological information monitored by the first sensor 12. For example, a drone, which is an example of the device 16, may be steered in accordance with the brain waves.

If it is determined based on data measured by the second sensor 14 that the first sensor 12 for monitoring brain waves is placed at a part other than the specific part where brain waves are monitored, the processor 24 does not operate the device 16 in accordance with the biological information monitored by the first sensor 12. That is, if the first sensor 12 is placed at a part other than the specific part, it is likely that biological information to be monitored by the first sensor 12 is biological information other than brain waves. Even when the first sensor 12 is not placed at the specific part, potentials or electric signals can still be measured, but they do not represent brain waves. In this case, the processor 24 does not operate the device 16 in accordance with the biological information monitored by the first sensor 12. If the first sensor 12 is not placed at the specific part of a user, information monitored by the first sensor 12 may not even be biological information concerning the user.

Processing to be executed by the processor 24 will be discussed below through illustration of another specific example. If the subject device 16 is a device which is assumed to be operated based on the amount of perspiration or myoelectric waveforms of the abdomen, the specific biological information is the amount of perspiration or myoelectric waveforms of the abdomen, and the specific part is the abdomen.

If it is determined based on data measured by the second sensor 14 that the first sensor 12 for monitoring the amount of perspiration or myoelectric waveforms is placed on the abdomen, the processor 24 operates the device 16 in accordance with the biological information monitored by the first sensor 12. For example, the vibration level of health equipment having a vibrating function, which is an example of the device 16, may be adjusted in accordance with the amount of perspiration or myoelectric waveforms of the abdomen.

If it is determined based on data measured by the second sensor 14 that the first sensor 12 for monitoring the amount of perspiration or myoelectric waveforms is placed at a part other than the abdomen, the processor 24 does not operate the device 16 in accordance with the biological information monitored by the first sensor 12.

A specific example of the management information will be explained below.

FIG. 3 illustrates an example of a management table, which is an example of the management information. Data in the management table is stored in the storage unit 22. Instead of in the storage unit 22, data in the management table may be stored in another apparatus (such as a server) other than the information processing apparatus 10.

In the management table, the ID, reference brain waves, and operation information indicating the operation content of the device 16 are linked with each other. Reference brain waves are an example of reference biological information. Biological information other than brain waves may be used.

The ID is information for managing and identifying a corresponding item of information registered in the management table.

The reference brain waves are determined by executing statistical processing, for example. The reference brain waves are brain waves that are assumed to be typically generated from a user who performs the operation linked with the reference brain waves or brain waves that are assumed to be generated from a user who makes a request to perform this operation.

The reference brain waves may be brain waves in a specific frequency band or may include brain waves in multiple frequency bands.

The operation information includes device identification information for identifying a device 16 to be operated and information indicating the content of operation to be performed on the device 16. For example, the content of operation may be the operation for turning ON or OFF the power supply of the device 16 or the operation for setting a function level of the device 16.

The reference brain waves of the ID “1” are those associated with the content of operation, namely, turning ON the cooling function of the air conditioner. The reference brain waves of the ID “2” are those associated with the content of operation, namely, turning OFF the cooling function of the air conditioner.

For example, if brain waves deviating from the reference brain waves of the ID “1” by an amount within the permissible range are monitored, the processor 24 identifies the content of operation indicated by the operation information linked with the reference brain waves, that is, the operation for turning ON the cooling function of the air conditioner. The processor 24 then sends control information including this operation information to the air conditioner. The air conditioner is operated in accordance with this control operation. That is, the cooling function of the air conditioner is turned ON.

The processor 24 may calculate the degree of similarity between the monitored brain waves concerning a user and the reference brain waves, and judge whether the calculated degree of similarity is higher than or equal to a threshold. The threshold corresponds to a value within the permissible range. If the degree of similarity between the monitored brain waves and the reference brain waves is higher than or equal to the threshold, the processor 24 determines that the monitored brain waves and the reference brain waves are similar to each other. That is, the processor 24 judges that the difference between the monitored brain waves and the reference brain waves is contained within the permissible range. If the degree of similarity between the monitored brain waves and the reference brain waves of the ID “1” is higher than or equal to the threshold, the processor 24 identifies the content of operation indicated by the operation information, that is, the operation for turning ON the cooling function of the air conditioner.

For each user, the reference biological information and operation information may be linked with each other and be registered in the management table. For example, monitored biological information concerning a user may be registered in the management table as reference biological information concerning this user.

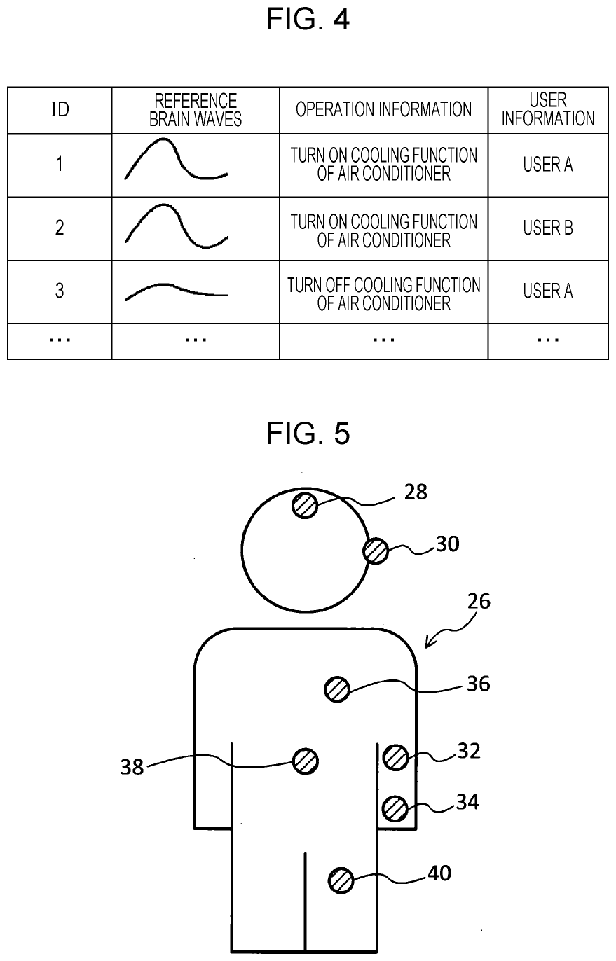

FIG. 4 illustrates an example of the management table in which items of specific reference biological information concerning individual users are registered. In the management table shown in FIG. 4, the ID, reference brain waves, which are an example of the reference biological information, operation information, and user information are linked with each other. The user information is information for identifying a user, such as the user name or user ID.

The reference brain waves linked with certain user information are brain waves which concern the user indicated by the user information and which are monitored when this user has performed the content of operation linked with the reference brain waves, or brain waves which are monitored when this user has made a request to perform the content of operation linked with the reference brain waves. Each set of monitored brain waves concerning a user are monitored in advance and are registered in the management table.

For example, when a user A has manually turned ON the cooling function of the air conditioner, the brain waves of the user A are monitored by the first sensor 12 and are registered in the management table as the reference brain waves associated with the content of operation “turning ON the cooling function of the air conditioner”.

In this case, the reference brain waves, operation information indicating the content of operation “turning ON the cooling function of the air conditioner”, user information for identifying the user A are linked with each other and are registered in the management table. These items of information may be registered by the information processing apparatus 10 or another apparatus. In the example shown in FIG. 4, these items of information are registered as those of the ID “1”. Information about brain waves concerning another user, information about the content of another operation, and information about this user are registered in a similar manner.

Brain waves concerning a user may be monitored multiple times, and then, the average brain waves may be registered as the reference brain waves. For example, the brain waves generated from the user A when the user A has manually turned ON the cooling function of the air conditioner are monitored by the first sensor 12 multiple times, and then, the average brain waves may be registered in the management table as the reference brain waves of the user A.

For example, when the user A is logging in the information processing apparatus 10, if brain waves concerning the user A deviating from the reference brain waves of the ID “1” by an amount within the permissible range are monitored, the processor 24 sends control information including the operation information of the ID “1” to the air conditioner so as to turn ON the cooling function of the air conditioner. This will be explained more specifically. While the user A is logging in the information processing apparatus 10, when brain waves concerning the user A are monitored by the first sensor 12, the processor 24 searches for the reference brain waves registered in the management table linked with the user information concerning the user A. In the example shown in FIG. 4, the reference brain waves of the ID “1” and those of the ID “3” are registered in the management table as the reference brain waves of the user A. If the difference between the monitored brain waves and the reference brain waves of the ID “1” is within the permissible range, the processor 24 sends control information including the operation information of the ID “1” to the air conditioner so as to turn ON the cooling function of the air conditioner. If the difference between the monitored brain waves and the reference brain waves of the ID “3” is within the permissible range, the processor 24 sends control information including the operation information of the ID “3” to the air conditioner so as to turn OFF the cooling function of the air conditioner.

In another example, in the state in which information that the user operating the device 16 is the user A is set in the information processing apparatus 10, if brain waves concerning the user A deviating from the reference brain waves of the ID “1” by an amount within the permissible range are monitored, the processor 24 may send control information including the operation information of the ID “1” to the air conditioner so as to turn ON the cooling function of the air conditioner. This will be explained more specifically. In the state in which information that the user operating the device 16 is the user A is set in the information processing apparatus 10, when the brain waves are monitored by the first sensor 12, the processor 24 searches for the reference brain waves linked with the user information concerning the user A and registered in the management table. If the difference between the monitored brain waves and the reference brain waves of the ID “1” is within the permissible range, the processor 24 sends control information including the operation information of the ID “1” to the air conditioner so as to turn ON the cooling function of the air conditioner. The user operating the device 16 may be set in the information processing apparatus 10 by this user, for example.

Concerning a user other than the user A, individual items of information are registered in the management table similarly. For example, individual items of information linked with the ID “2” are those concerning the operation when the user B has turned ON the cooling function of the air conditioner. Individual items of information linked with the ID “3” are those concerning the operation when the user A has turned OFF the cooling function of the air conditioner.

In the management tables shown in FIGS. 3 and 4, operation information indicating the operation for turning ON or OFF the power supply of the device 16 is registered. Alternatively, operation information indicating a function level of the device 16 may be registered in the management table.

Parts of the body where the first sensor 12 may be placed will be explained below with reference to FIG. 5. FIG. 5 illustrates a schematic model of a human body 26.

For example, the first sensor 12 may be placed at the forehead 28, ear 30, arm 32, hand 34, chest 36, abdomen 38, or thigh 40 of the human body 26. The first sensor 12 may be placed at a part other than the above-described parts. The first sensor 12 may be placed at each of multiple parts.

For example, the first sensor 12 that monitors brain waves or the body temperature may be placed at the forehead 28 or the ear 30. The first sensor 12 that monitors myoelectric waveforms, blood flow, the amount of perspiration, body temperature, heart rate, or blood pressure may be placed on the arm 32, hand 34, or a wrist. The first sensor 12 that monitors electrocardiogram waveforms, heart rate, or body temperature may be placed on the chest 36. The first sensor 12 that monitors the amount of perspiration or myoelectric waveforms may be placed on the abdomen 38 or the thigh 40. The first sensor 12 that monitors another type of biological information may be placed on a corresponding part of the body.

In accordance with the type of specific biological information for operating the subject device 16, the specific part varies and the type of first sensor 12 for monitoring the specific biological information at the specific part also varies. For example, if the subject device 16 is a device assumed to be operated in accordance with brain waves, it is likely that the first sensor 12 which monitors brain waves is placed at a part where brain waves are monitored, such as at the forehead 28 or the ear 30. In this case, brain waves are the specific biological information, the forehead 28 or the ear 30 is the specific part, and the first sensor 12 monitoring brain waves is the first sensor 12 that monitors the specific biological information.

Examples of the exemplary embodiment will be described below.

### FIRST EXAMPLE

A first example will be discussed below.

In the first example, the second sensor 14 is a temperature sensor which measures the temperature of a part of a user where the first sensor 12 is placed. It is judged whether the first sensor 12 is placed on a specific part, based on the temperature measured by the temperature sensor.

In one example, the second sensor 14 may be attached to the first sensor 12 and be placed at a part of a user together with the first sensor 12 so as to measure the temperature of the part where the first sensor 12 is placed. In another example, the second sensor 14 may be located separately from the first sensor 12, for example, it is located near the first sensor 12 so as to be placed on a part of a user, thereby measuring the temperature of the part where the first sensor 12 is placed. In another example, the second sensor 14 may not be placed at any part of a user and measure the temperature of the part where the first sensor 12 is placed.

Temperature data indicating the temperature measured by the second sensor 14 is sent from the second sensor 14 to the information processing apparatus 10. The temperature data is data indicating the temperature of the part where the first sensor 12 is placed. The processor 24 receives the temperature data sent from the second sensor 14 so as to identify the temperature of the part where the first sensor 12 is placed.

The processor 24 judges whether the first sensor 12 is placed at the specific part, based on the temperature indicated by the temperature data.

If the temperature indicated by the temperature data is included within a range of temperatures of the specific part related to a subject device 16 to be operated, the processor 24 judges that the first sensor 12 is placed at the specific part. That is, if the temperature indicated by the temperature data is included within the range of temperatures of the specific part where the first sensor 12, which monitors the specific biological information for operating the device 16, is assumed to be placed, the processor 24 judges that the first sensor 12 is placed at the specific part. If the temperature indicated by the temperature data is included within the range of temperatures of the specific part, it is likely that the biological information monitored by the first sensor 12 is the specific biological information for operating the device 16. In this case, the processor 24 operates the device 16 in accordance with the biological information monitored by the first sensor 12.

If the temperature indicated by the temperature data is not included within the range of temperatures of the specific part, the processor 24 judges that the first sensor 12 is not placed at the specific part. If the temperature indicated by the temperature data is not included within the range of temperatures of the specific part, it is unlikely that the biological information monitored by the first sensor 12 is the specific biological information for operating the device 16. In this case, the processor 24 does not operate the device 16 in accordance with the biological information monitored by the first sensor 12.

It is known that the body temperature varies according to the part of the human body. Using the difference in the body temperature makes it possible to judge whether the first sensor 12 is placed at the specific part. For example, a range of temperatures is set for each part of the human body in advance, and a range of temperatures of the specific part is also determined in advance. Information indicating the range of temperatures of each part is stored in the storage unit 22 or another device (such as a server). The range of temperatures for each part may be a range of actually measured temperatures or that determined by executing statistical processing.

The body temperature may vary according to the environments (the season, the location, such as the indoor location and the outdoor location, and the operating conditions of the air conditioner, for example). Hence, the range of temperatures for each part may be changed in accordance with the environments.

Details of the first example will be discussed below through illustration of a specific example.

The subject device 16 is a device which is assumed to be operated in accordance with brain waves, for example. That is, the specific biological information is brain waves.

The first sensor 12 is a sensor which monitors brain waves, which are an example of the specific biological information for operating the device 16. The specific part is the head (such as the forehead or the ear) of a user, for example. That is, the first sensor 12 is a sensor which is assumed to be fixed on the head of the user (such as the forehead or the ear). The first sensor 12 is attached to the forehead or the ear (ear canal, for example) so as to monitor brain waves.

The processor 24 judges whether the first sensor 12 is fixed on the head (such as the forehead or the ear) of a user, based on the temperature indicated by the temperature data. That is, the processor 24 judges whether the first sensor 12 is placed at the part where brain waves can be monitored, based on the temperature indicated by the temperature data.

If the temperature indicated by the temperature data is included within the range of temperatures of the ear or the forehead of a user, the processor 24 determines that the first sensor 12 is fixed on the head of the user. That is, the processor 24 determines that the first sensor 12 is placed at the part where brain waves can be monitored.

For example, the range of temperatures of the ear (ear canal, for example) and that of the forehead are determined in advance. These ranges of temperatures may be those of actually measured temperatures or those determined by executing statistical processing. If the temperature measured by the second sensor 14 is included within the range of temperatures of the ear, the processor 24 judges that the first sensor 12 is fixed at the ear of the user. If the temperature measured by the second sensor 14 is included within the range of temperatures of the forehead, the processor 24 judges that the first sensor 12 is fixed on the forehead of the user. The range of temperatures of the ear and that of the forehead may overlap each other. In this case, instead of determining whether the first sensor 12 is placed at the ear or on the forehead, the processor 24 may simply judge that the first sensor 12 is fixed on the head of the user.

If the first sensor 12 is inserted into the ear canal, the temperature of a portion with the first sensor 12 may become higher. It is assumed, for example, that brain waves are monitored by a bearable device, such as an earphone. The first sensor 12 is attached to an earpiece of the earphone, and the earpiece is inserted into the ear canal. Then, the ear canal is sealed by the earpiece, and the temperature of the part where the first sensor 12 is placed may be raised. The second sensor 14 may also be attached to the earpiece so as to measure the temperature of the part (inside the ear, for example) where the first sensor 12 is placed. In contrast to the ear canal, the forehead is exposed to outside air. Hence, the temperature measured by the second sensor 14 when the first sensor 12 is fit in the ear canal can be higher than that when the first sensor 12 is placed on the forehead. Based on this temperature difference, the processor 24 may determine whether the first sensor 12 is placed in the ear canal or on the forehead. If the temperature measured by the second sensor 14 is higher than or equal to a temperature threshold, the processor 24 may determine that the first sensor 12 is placed in the ear canal. If the temperature measured by the second sensor 14 is lower than the temperature threshold, the processor 24 may determine that the first sensor 12 is fixed on the forehead.

Since the normal body temperature is different among users, the range of temperatures of the specific part may be determined for each user. For example, for each user, the range of temperatures of the ear and that of the forehead may be determined.

If the temperature measured by the second sensor 14 is included within the range of temperatures of the ear or that of the forehead, it is likely that the first sensor 12 is placed on the head (such as the ear or the forehead). That is, it is likely that the biological information monitored by the first sensor 12 is brain waves. In this case, the processor 24 operates the device 16 in accordance with the biological information monitored by the first sensor 12.

If the temperature measured by the second sensor 14 is not included within the range of temperatures of the ear or that of the forehead, it is likely that the first sensor 12 is placed at a part other than the head (such as the ear or the forehead). That is, it is unlikely that the biological information monitored by the first sensor 12 is brain waves. In this case, the processor 24 does not operate the device 16 in accordance with the biological information monitored by the first sensor 12.

As described above, when the first sensor 12, which monitors brain waves for operating the device 16, is placed on the head (such as the ear or the forehead), which is the specific part where brain waves can be monitored, the device 16 can be operated.

The device 16 according to the first example is a device assumed to be operated in accordance with brain waves. The device 16 in the first example may alternatively be a device operated in accordance with biological information other than brain waves. For example, the device 16 may be a device operated in accordance with the pulse rate or the blood flow. In this case, the first sensor 12 is a sensor which monitors the pulse rate or the blood flow. For example, the first sensor 12 is placed in the ear canal to monitor the pulse rate or the blood flow. If the temperature measured by the second sensor 14 is included within the range of temperatures of the ear canal, the processor 24 judges that the first sensor 12 is placed in the ear canal. The processor 24 then operates the device 16 in accordance with the pulse rate or the blood flow monitored by the first sensor 12.

### SECOND EXAMPLE

A second example will be discussed below.

In the second example, the second sensor 14 is a pressure sensor which measures the pressure level of a part of a user where the first sensor 12 is placed. It is judged whether the first sensor 12 is placed at a specific part, based on the pressure level measured by the pressure sensor.

In one example, the second sensor 14 may be attached to the first sensor 12 and be placed at a part of a user together with the first sensor 12 so as to measure the pressure level of the part where the first sensor 12 is placed. In another example, the second sensor 14 may be located separately from the first sensor 12, for example, it is located near the first sensor 12 so as to be placed at a part of a user, thereby measuring the pressure level of the part where the first sensor 12 is placed.

Pressure data indicating the pressure level measured by the second sensor 14 is sent from the second sensor 14 to the information processing apparatus 10. The pressure data is data indicating the pressure level of the part where the first sensor 12 is placed. The processor 24 receives the pressure data sent from the second sensor 14 so as to identify the pressure level of the part where the first sensor 12 is placed.

The processor 24 judges whether the first sensor 12 is placed at the specific part, based on the pressure level indicated by the pressure data.

If the pressure level indicated by the pressure data is included within a range of pressure levels of the specific part related to a subject device 16 to be operated, the processor 24 judges that the first sensor 12 is placed at the specific part. That is, if the pressure level indicated by the pressure data is included within the range of pressure levels of the specific part where the first sensor 12, which monitors the specific biological information for operating the device 16, is assumed to be placed, the processor 24 judges that the first sensor 12 is placed at the specific part. If the pressure level indicated by the pressure data is included within the range of pressure levels of the specific part, it is likely that the biological information monitored by the first sensor 12 is the specific biological information for operating the device 16. In this case, the processor 24 operates the device 16 in accordance with the biological information monitored by the first sensor 12.

If the pressure level indicated by the pressure data is not included within the range of pressure levels of the specific part, the processor 24 judges that the first sensor 12 is not placed at the specific part. If the pressure level indicated by the pressure data is not included within the range of pressure levels of the specific part, it is unlikely that the biological information monitored by the first sensor 12 is the specific biological information for operating the device 16. In this case, the processor 24 does not operate the device 16 in accordance with the biological information monitored by the first sensor 12.

For example, a range of pressure levels is set for each part of the human body in advance, and a range of pressure levels of the specific part is also determined in advance. Information indicating the range of pressure levels of each part is stored in the storage unit 22 or another device (such as a server). The range of pressure levels for each part may be a range of actually measured pressure levels or that determined by executing statistical processing.

Details of the second example will be discussed below through illustration of a specific example.

The subject device 16 is a device which is assumed to be operated in accordance with brain waves, for example.

The first sensor 12 is a sensor which monitors brain waves, which are an example of the specific biological information for operating the device 16. The specific part is the ear (such as the ear canal) of a user, for example. That is, the first sensor 12 is a sensor assumed to be placed in the ear (ear canal, for example). The first sensor 12 is placed in the ear (ear canal, for example) so as to monitor brain waves.

Brain waves are monitored by a bearable device, such as an earphone, for example. The first sensor 12 is attached to an earpiece of the earphone, and the earpiece is inserted into the ear canal. Then, the first sensor 12 can monitor brain waves. The second sensor 14 may also be attached to the earpiece so as to measure the pressure level of the part (inside the ear, for example) where the first sensor 12 is placed.

The processor 24 judges whether the first sensor 12 is placed in the ear canal of a user, based on the pressure level indicated by the pressure data. That is, the processor 24 judges whether the first sensor 12 is placed at a position where brain waves can be monitored, based on the pressure level indicated by the pressure data.

For example, if the pressure level indicated by the pressure data is higher than or equal to a pressure threshold, the processor 24 determines that the first sensor 12 is placed in the ear canal of the user. That is, the processor 24 determines that the first sensor 12 is placed at a position where brain waves can be monitored.

When the first sensor 12 is inserted into the ear canal, the pressure level of a portion with the first sensor 12 may become higher. For example, the first sensor 12 is inserted into the earpiece of the earphone, and the earpiece is inserted into the ear canal. Then, the inside of the ear is sealed by the earpiece, and the pressure level of the inside of the ear may become higher than that outside the ear. When the inside of the ear is sealed by the earpiece, air within the ear is compressed, which may raise the pressure level within the ear.

The pressure threshold is determined by taking a rise in the pressure level into account. For example, the pressure threshold is set to be a value higher than the pressure level outside the ear and lower than the raised pressure level inside the ear.

The pressure level inside the ear may be different among users. Hence, the pressure threshold may be determined for each user.

If the pressure level measured by the second sensor 14 is higher than or equal to the pressure threshold, it is likely that the first sensor 12 is placed in the ear canal. That is, it is likely that the biological information monitored by the first sensor 12 is brain waves monitored in the ear canal. In this case, the processor 24 operates the device 16 in accordance with the biological information monitored by the first sensor 12.

If the pressure level measured by the second sensor 14 is lower than the threshold, it is likely that the first sensor 12 is placed at a part other than the ear canal. That is, it is unlikely that the biological information monitored by the first sensor 12 is brain waves monitored in the ear canal. In this case, the processor 24 does not operate the device 16 in accordance with the biological information monitored by the first sensor 12.

As described above, when the first sensor 12, which monitors brain waves for operating the device 16, is placed in the ear canal, which is the specific part where brain waves can be monitored, the device 16 can be operated.

The first example and the second example may be combined with each other. In this case, a temperature sensor and a pressure sensor are used as the second sensor 14. If the temperature measured by the temperature sensor is included within the range of temperatures of the specific part of the user (the ear canal, for example) and if the pressure level measured by the pressure sensor is included within the range of pressure levels of the specific part (for example, if the pressure level is higher than or equal to the pressure threshold), the processor 24 judges that the first sensor 12 is placed at the specific part (the ear canal, for example) and operates the device 16 in accordance with the biological information monitored by the first sensor 12. If the above-described conditions are not satisfied, the processor 24 judges that the first sensor 12 is placed at a part other than the specific part and does not operate the device 16 in accordance with the biological information monitored by the first sensor 12.

The device 16 according to the second example is a device assumed to be operated in accordance with brain waves. The device 16 in the second example may alternatively be a device operated in accordance with biological information other than brain waves. For example, the device 16 may be a device operated in accordance with the pulse rate or the blood flow, and the first sensor 12 may be a sensor which monitors the pulse rate or the blood flow.

### THIRD EXAMPLE

A third example will be discussed below.

In the third example, the second sensor 14 is an illuminance sensor which measures the illuminance level of a part of a user where the first sensor 12 is placed. It is judged whether the first sensor 12 is placed on a specific part, based on the illuminance level measured by the illuminance sensor.

In one example, the second sensor 14 may be attached to the first sensor 12 and be placed at a part of a user together with the first sensor 12 so as to measure the illuminance level of the part where the first sensor 12 is placed. In another example, the second sensor 14 may be located separately from the first sensor 12, for example, it is located near the first sensor 12 so as to be placed at a part of a user, thereby measuring the illuminance level of the part where the first sensor 12 is placed.

Illuminance data indicating the illuminance level measured by the second sensor 14 is sent from the second sensor 14 to the information processing apparatus 10. The illuminance data is data indicating the illuminance level of the part where the first sensor 12 is placed. The processor 24 receives the illuminance data sent from the second sensor 14 so as to identify the illuminance level of the part where the first sensor 12 is placed.

The processor 24 judges whether the first sensor 12 is placed at the specific part, based on the illuminance level indicated by the illuminance data.

If the illuminance level indicated by the illuminance data is included within a range of illuminance levels of the specific part related to a subject device 16 to be operated, the processor 24 judges that the first sensor 12 is placed at the specific part. That is, if the illuminance level indicated by the illuminance data is included within the range of illuminance levels of the specific part where the first sensor 12, which monitors specific biological information for operating the device 16, is assumed to be placed, the processor 24 judges that the first sensor 12 is placed at the specific part. If the illuminance level indicated by the illuminance data is included within the range of illuminance levels of the specific part, it is likely that the biological information monitored by the first sensor 12 is the specific biological information for operating the device 16. In this case, the processor 24 operates the device 16 in accordance with the biological information monitored by the first sensor 12.

If the illuminance level indicated by the illuminance data is not included within the range of illuminance levels of the specific part, the processor 24 judges that the first sensor 12 is not placed at the specific part. If the illuminance level indicated by the illuminance data is not included within the range of illuminance levels of the specific part, it is unlikely that the biological information monitored by the first sensor 12 is the specific biological information for operating the device 16. In this case, the processor 24 does not operate the device 16 in accordance with the biological information monitored by the first sensor 12.

For example, a range of illuminance levels is set for each part of the human body in advance, and a range of illuminance levels of the specific part is also determined in advance. Information indicating the range of illuminance levels of each part is stored in the storage unit 22 or another device (such as a server). The range of illuminance levels for each part may be a range of actually measured illuminance levels or that determined by executing statistical processing.

Details of the third example will be discussed below through illustration of a specific example.

The subject device 16 is a device which is assumed to be operated in accordance with brain waves, for example.

The first sensor 12 is a sensor which monitors brain waves, which are an example of the specific biological information for operating the device 16. The specific part is the ear (such as the ear canal) of a user, for example. That is, the first sensor 12 is a sensor assumed to be placed in the ear (ear canal, for example). The first sensor 12 is placed in the ear (ear canal, for example) so as to monitor brain waves.

For example, brain waves are monitored by a bearable device, such as an earphone. The first sensor 12 is attached to an earpiece of the earphone, and the earpiece is inserted into the ear canal. Then, the first sensor 12 can monitor brain waves. The second sensor 14 may also be attached to the earpiece so as to measure the illuminance of the part (inside the ear, for example) where the first sensor 12 is placed.

The processor 24 judges whether the first sensor 12 is placed in the ear canal of a user, based on the illuminance level indicated by the illuminance data. That is, the processor 24 judges whether the first sensor 12 is placed at a position where brain waves can be monitored, based on the illuminance level indicated by the illuminance data.

For example, if the illuminance level indicated by the illuminance data is lower than or equal to an illuminance threshold, the processor 24 determines that the first sensor 12 is placed in the ear canal of the user. That is, the processor 24 determines that the first sensor 12 is placed at a position where brain waves can be monitored.

When the first sensor 12 is inserted into the ear canal, the portion with the first sensor 12 becomes darker than outside the ear. For example, the first sensor 12 is attached to the earpiece of the earphone, and the earpiece is inserted into the ear canal. Then, the inside of the ear is sealed by the earpiece and becomes darker than outside the ear.

The illuminance threshold is determined by taking the illuminance level inside the ear into account. For example, the illuminance threshold is set to be the level lower than that outside the ear and higher than that inside the ear, for example.

The illuminance level inside the ear may be different among users. Hence, the illuminance threshold may be determined for each user.

If the illuminance level measured by the second sensor 14 is lower than or equal to the illuminance threshold, it is likely that the first sensor 12 is placed in the ear canal. That is, it is likely that the biological information monitored by the first sensor 12 is brain waves monitored in the ear canal. In this case, the processor 24 operates the device 16 in accordance with the biological information monitored by the first sensor 12.

If the illuminance level measured by the second sensor 14 exceeds the illuminance threshold, it is likely that the first sensor 12 is placed at a part other than the ear canal. That is, it is unlikely that the biological information monitored by the first sensor 12 is brain waves monitored in the ear canal. In this case, the processor 24 does not operate the device 16 in accordance with the biological information monitored by the first sensor 12.

As described above, when the first sensor 12, which monitors brain waves for operating the device 16, is placed in the ear canal, which is the specific part where brain waves can be monitored, the device 16 can be operated.

The first example and the third example may be combined with each other. In this case, a temperature sensor and an illuminance sensor are used as the second sensor 14. If the temperature measured by the temperature sensor is included within the range of temperatures of the specific part of the user (the ear canal, for example) and if the illuminance level measured by the illuminance sensor is included within the range of illuminance levels of the specific part (for example, if the illuminance level is lower than or equal to the illuminance threshold), the processor 24 judges that the first sensor 12 is placed at the specific part (the ear canal, for example) and operates the device 16 in accordance with the biological information monitored by the first sensor 12. If the above-described conditions are not satisfied, the processor 24 judges that the first sensor 12 is placed at a part other than the specific part and does not operate the device 16 in accordance with the biological information monitored by the first sensor 12.

The second example and the third example may be combined with each other. In this case, a pressure sensor and an illuminance sensor are used as the second sensor 14. If the pressure level measured by the pressure sensor is included within the range of pressure levels of the specific part (for example, if the pressure level is higher than or equal to the pressure threshold) and if the illuminance level measured by the illuminance sensor is included within the range of illuminance levels of the specific part (for example, if the illuminance level is lower than or equal to the illuminance threshold), the processor 24 judges that the first sensor 12 is placed at the specific part (the ear canal, for example) and operates the device 16 in accordance with the biological information monitored by the first sensor 12. If the above-described conditions are not satisfied, the processor 24 judges that the first sensor 12 is placed at a part other than the specific part and does not operate the device 16 in accordance with the biological information monitored by the first sensor 12.

The first example, the second example, and the third example may be combined with each other. In this case, a temperature sensor, a pressure sensor, and an illuminance sensor are used as the second sensor 14. If the temperature measured by the temperature sensor is included within the range of temperatures of the specific part of the user (the ear canal, for example), and if the pressure level measured by the pressure sensor is included within the range of pressure levels of the specific part (for example, and if the pressure level is higher than or equal to the pressure threshold), and if the illuminance level measured by the illuminance sensor is included within the range of illuminance levels of the specific part (for example, if the illuminance level is lower than or equal to the illuminance threshold), the processor 24 judges that the first sensor 12 is placed at the specific part (the ear canal, for example) and operates the device 16 in accordance with the biological information monitored by the first sensor 12. If the above-described conditions are not satisfied, the processor 24 judges that the first sensor 12 is placed at a part other than the specific part and does not operate the device 16 in accordance with the biological information monitored by the first sensor 12.

The device 16 according to the third example is a device assumed to be operated in accordance with brain waves. The device 16 in the third example may alternatively be a device operated in accordance with biological information other than brain waves. For example, the device 16 may be a device operated in accordance with the pulse rate or the blood flow, and the first sensor 12 may be a sensor which monitors the pulse rate or the blood flow.

### FOURTH EXAMPLE

A fourth example will be discussed below.

In the fourth example, the second sensor 14 is a motion sensor which measures the amount of movement of a part of a user where the first sensor 12 is placed. Examples of the motion sensor are a gyroscope and an acceleration sensor. It is judged whether the first sensor 12 is placed at a specific part, based on the amount of movement measured by the motion sensor. The amount of movement of a part is the amount of positional change, the rate of positional change, the moving speed, the moving acceleration, the amount of vibration, or a combination thereof. The amount of movement may include information indicating the moving direction of a part.

In one example, the second sensor 14 may be attached to the first sensor 12 and be placed at a part of a user together with the first sensor 12 so as to measure the amount of movement of the part where the first sensor 12 is placed. In another example, the second sensor 14 may be located separately from the first sensor 12, for example, it is located near the first sensor 12 so as to be placed at a part of a user, thereby measuring the amount of movement of the part where the first sensor 12 is placed.

Motion data indicating the amount of movement measured by the second sensor 14 is sent from the second sensor 14 to the information processing apparatus 10. The motion data is data indicating the amount of movement of the part where the first sensor 12 is placed. The processor 24 receives the motion data sent from the second sensor 14 so as to identify the amount of movement of the part where the first sensor 12 is placed.

The processor 24 judges whether the first sensor 12 is placed at the specific part, based on the amount of movement indicated by the motion data.

If the amount of movement indicated by the motion data is included within a range of the amounts of movement of the specific part related to a subject device 16 to be operated, the processor 24 judges that the first sensor 12 is placed at the specific part. That is, if the amount of movement indicated by the motion data is included within the range of the amounts of movement of the specific part where the first sensor 12, which monitors specific biological information for operating the device 16, is assumed to be placed, the processor 24 judges that the first sensor 12 is placed at the specific part. If the amount of movement indicated by the motion data is included within the range of the amounts of movement of the specific part, it is likely that the biological information monitored by the first sensor 12 is the specific biological information for operating the device 16. In this case, the processor 24 operates the device 16 in accordance with the biological information monitored by the first sensor 12.

If the amount of movement indicated by the motion data is not included within the range of the amounts of movement of the specific part, the processor 24 judges that the first sensor 12 is not placed at the specific part. If the amount of movement indicated by the motion data is not included within the range of the amounts of movement of the specific part, it is unlikely that the biological information monitored by the first sensor 12 is the specific biological information for operating the device 16. In this case, the processor 24 does not operate the device 16 in accordance with the biological information monitored by the first sensor 12.

The amount of movement may vary according to the part of the human body. Using the difference in the amount of movement makes it possible to judge whether the first sensor 12 is placed at the specific part. For example, a range of the amounts of movement is set for each part of the human body in advance, and a range of the amounts of movement of the specific part is also determined in advance. Information indicating the range of the amounts of movement of each part is stored in the storage unit 22 or another device (such as a server). The range of the amounts of movement for each part may be a range of actually measured amounts of movement or that determined by executing statistical processing.

Characteristics of the movement may be determined for each part of the human body in advance. Information indicating the characteristics of the movement of each part is stored in the storage unit 22 or another device (such as a server). The processor 24 may extract the characteristics of the movement from the measurement results obtained by the second sensor 14 and compare the extracted characteristics with the characteristics of the movement of each part, thereby determining whether the first sensor 12 is placed at the specific part.

Details of the fourth example will be discussed below through illustration of a specific example.

The subject device 16 is a device which is assumed to be operated in accordance with brain waves, for example.

The first sensor 12 is a sensor which monitors brain waves, which are an example of the specific biological information for operating the device 16. The specific part is the head (such as the forehead or the ear) of a user, for example. That is, the first sensor 12 is a sensor assumed to be placed on the head (such as the forehead or the ear). The first sensor 12 is placed on the head (such as the forehead or the ear) so as to monitor brain waves. The first sensor 12 may be attached to the earpiece of an earphone, and the earpiece may be inserted into the ear canal, for example.

The processor 24 judges whether the first sensor 12 is placed on the head (such as the forehead or the ear), based on the amount of movement indicated by the motion data. That is, the processor 24 judges whether the first sensor 12 is placed at a position where brain waves can be monitored, based on the amount of movement indicated by the motion data.

For example, if the amount of movement indicated by the motion data is included within a range of the amounts of movement of the head of a user, the processor 24 determines that the first sensor 12 is placed on the head (such as the forehead or the ear) of the user. That is, the processor 24 determines that the first sensor 12 is placed at a position where brain waves can be monitored.

The range of the amounts of movement of the head is determined in advance. Usually, the amount of movement of the head is smaller than that of the arm or the wrist, for example. Regarding a positional change of the head, the amount of change, the moving speed, the moving acceleration, the amount of vibration are likely to be smaller than those of the arm or the wrist, for example. The range of the amounts of movement of the head is determined by taking the above-described factors into account.

The range of the amounts of movement of the head may be different among users. Hence, the range of the amounts of movement of the head may be determined for each user.

The processor 24 may extract the characteristics of the movement from the measurement results obtained by the second sensor 14 and compare the extracted characteristics with the characteristics of the movement of the head, thereby determining whether the first sensor 12 is placed on the head.

If the amount of movement measured by the second sensor 14 is included within the range of the amounts of movement of the head, it is likely that the first sensor 12 is placed on the head. That is, it is likely that the biological information monitored by the first sensor 12 is brain waves. In this case, the processor 24 operates the device 16 in accordance with the biological information monitored by the first sensor 12.

If the amount of movement measured by the second sensor 14 is not included within the range of the amounts of movement of the head, it is likely that the first sensor 12 is placed at a part other than the head. That is, it is unlikely that the biological information monitored by the first sensor 12 is brain waves. In this case, the processor 24 does not operate the device 16 in accordance with the biological information monitored by the first sensor 12.

As described above, when the first sensor 12, which monitors brain waves for operating the device 16, is placed on the head, which is the specific part where brain waves can be monitored, the device 16 can be operated.

The fourth example may be combined with at least one of the first, second, and third examples.

### FIFTH EXAMPLE

A fifth example will be discussed below.

In the fifth example, the second sensor 14 is an odor sensor which measures the odor level of a part of a user where the first sensor 12 is placed. The odor index or the odor intensity, for example, is measured by the odor sensor.

In one example, the second sensor 14 may be attached to the first sensor 12 and be placed at a part of a user together with the first sensor 12 so as to measure the odor level of the part where the first sensor 12 is placed. In another example, the second sensor 14 may be located separately from the first sensor 12, for example, it is located near the first sensor 12 so as to be placed at a part of a user, thereby measuring the odor level of the part where the first sensor 12 is placed.

Odor data indicating the odor level measured by the second sensor 14 is sent from the second sensor 14 to the information processing apparatus 10. The odor data is data indicating the odor level of the part where the first sensor 12 is placed. The processor 24 receives the odor data sent from the second sensor 14 so as to identify the odor level of the part where the first sensor 12 is placed.

The processor 24 judges whether the first sensor 12 is placed at the specific part, based on the odor level indicated by the odor data.

If the odor level (such as the odor index or the odor intensity) indicated by the odor data is included within a range of odor levels (such as odor index values or odor intensity values) of the specific part related to a subject device 16 to be operated, the processor 24 judges that the first sensor 12 is placed at the specific part. That is, if the odor level indicated by the odor data is included within the range of odor levels of the specific part where the first sensor 12, which monitors specific biological information for operating the device 16, is assumed to be placed, the processor 24 judges that the first sensor 12 is placed at the specific part. If the odor level indicated by the odor data is included within the range of odor levels of the specific part, it is likely that the biological information monitored by the first sensor 12 is the specific biological information for operating the device 16. In this case, the processor 24 operates the device 16 in accordance with the biological information monitored by the first sensor 12.

If the odor level indicated by the odor data is not included within the range of odor levels of the specific part, the processor 24 judges that the first sensor 12 is not placed at the specific part. If the odor level indicated by the odor data is not included within the range of odor levels of the specific part, it is unlikely that the biological information monitored by the first sensor 12 is the specific biological information for operating the device 16. In this case, the processor 24 does not operate the device 16 in accordance with the biological information monitored by the first sensor 12.

The odor level may vary according to the part of the human body. The odor level may also be different between the part exposed to outside air and the part which is not exposed to outside air. For example, the odor of a portion covered with clothes may be different from that of a portion without clothes. This will be explained by way of a specific example. Usually, the head (the forehead, for example) is not covered with clothes and is exposed to outside air. The odor level of the head is different from that of the part, such as the chest, abdomen, and lower limb. Usually, the odor level of the part exposed to outside air is weaker than that of the part which is not exposed to outside air. For example, the odor index or the odor intensity of the part exposed to outside air is lower than that of the part which is not exposed to outside air. In this manner, the odor level may be different according to the part. A range of odor levels is set for each part of the human body in advance, and a range of odor levels of the specific part is also determined in advance.

Information indicating the range of odor levels of each part is stored in the storage unit 22 or another device (such as a server).

Details of the fifth example will be discussed below through illustration of a specific example.

The subject device 16 is a device which is assumed to be operated in accordance with brain waves, for example.

The first sensor 12 is a sensor which monitors brain waves, which are an example of the specific biological information for operating the device 16. The specific part is the head (the forehead, for example) of a user, for example. That is, the first sensor 12 is a sensor assumed to be placed on the head (such as the forehead or the ear). The first sensor 12 is placed on the head so as to monitor brain waves.

The processor 24 judges whether the first sensor 12 is placed on the head (the forehead, for example), based on the odor level indicated by the odor data. That is, the processor 24 judges whether the first sensor 12 is placed at a position where brain waves can be monitored, based on the odor level indicated by the odor data.

For example, if the odor level indicated by the odor data is lower than or equal to an odor threshold, the processor 24 determines that the first sensor 12 is placed on the head (the forehead, for example) of the user. That is, the processor 24 determines that the first sensor 12 is placed at a position where brain waves can be monitored.

The odor threshold is determined by taking the fact that the head is exposed to outside air into account. For example, the odor threshold is determined so that the odor level of a part exposed to outside air becomes lower than or equal to the odor threshold.

The odor level may be different among users. Hence, the odor threshold may be determined for each user.

If the odor level measured by the second sensor 14 is lower than or equal to the odor threshold, it is likely that the first sensor 12 is placed on the head. That is, it is likely that the biological information monitored by the first sensor 12 is brain waves. In this case, the processor 24 operates the device 16 in accordance with the biological information monitored by the first sensor 12.

If the odor level measured by the second sensor 14 exceeds the odor threshold, it is likely that the first sensor 12 is placed at a part other than the head. That is, it is unlikely that the biological information monitored by the first sensor 12 is brain waves. In this case, the processor 24 does not operate the device 16 in accordance with the biological information monitored by the first sensor 12.

As described above, when the first sensor 12, which monitors brain waves for operating the device 16, is placed on the head, which is the specific part where brain waves can be monitored, the device 16 can be operated.

The fifth example may be combined with at least one of the first through fourth examples.

### SIXTH EXAMPLE

A sixth example will be described below.

In the sixth example, the second sensor 14 is an image capturing device installed in the first sensor 12. For example, the image capturing device performs an image capturing operation so as to generate image data indicating the range where the image is captured (hereinafter such a range will be called the image capturing range of the second sensor 14). The image data may be still image data or video image data.

The second sensor 14 is installed in the first sensor 12. Hence, upon analyzing image data generated by the image capturing operation of the second sensor 14, the part of a user where the first sensor 12 is placed is likely to be identified.

The image capturing range of the second sensor 14 placed on the forehead is different from that of the second sensor 14 placed on the arm. The image data generated by the image capturing operation of the second sensor 14 accordingly becomes different depending on the part where the first sensor 12 is placed.

For example, if the image capturing range of the second sensor 14 is included within a specific range corresponding to a specific part, the processor 24 determines that the first sensor 12 is placed at the specific part. In this case, the processor 24 operates the device 16 in accordance with the biological information monitored by the first sensor 12. The processor 24 judges whether the image capturing range of the second sensor 14 is included within the specific range related to the specific part, based on the image data generated by the image capturing operation of the second sensor 14.

If the image capturing range of the second sensor 14 is not included within the specific range, the processor 24 determines that the first sensor 12 is not placed at the specific part. In this case, the processor 24 does not operate the device 16 in accordance with the biological information monitored by the first sensor 12.

Details of the sixth example will be discussed below through illustration of a specific example.

The subject device 16 is a device which is assumed to be operated in accordance with brain waves, for example.

The first sensor 12 is a sensor which monitors brain waves, which are an example of the specific biological information for operating the device 16. The specific part is the head (the forehead, for example) of a user, for example. That is, the first sensor 12 is a sensor assumed to be placed on the head. The first sensor 12 is placed on the head so as to monitor brain waves.

If the image capturing range of the second sensor 14 is included within the range corresponding to the head, the processor 24 determines that the first sensor 12 is placed on the head (the forehead, for example) of the user. That is, if the image data is data generated as a result of capturing an image from the position of the head, the processor 24 determines that the first sensor 12 is placed on the head of the user. In this case, the processor 24 operates the device 16 in accordance with the biological information monitored by the first sensor 12.

If the image capturing range of the second sensor 14 is included within a range corresponding to a part (the arm, for example) other than the head, the processor 24 determines that the first sensor 12 is placed at a part other than the head. That is, if the image data is data generated as a result of capturing an image from the position of a part other than the head, the processor 24 determines that the first sensor 12 is placed at a part other than the head of the user. In this case, the processor 24 does not operate the device 16 in accordance with the biological information monitored by the first sensor 12.

The processor 24 may identify the part of a user where the first sensor 12 is placed, based on information indicating a portion of the user or a portion of clothes worn by the user included in an image captured by the second sensor 14 in the image capturing range. For example, if an image of a portion of the head (such as the cheek, mustache, beard, nose, and the side of the head) of the user is captured by the second sensor 14, that is, if the image data indicates a portion of the head of the user, the processor 24 determines that the first sensor 12 is placed on the head of the user. If an image of a portion of the user's hand or wrist or user's clothes (sleeve, for example) is captured by the second sensor 14, that is, if the image data indicates a portion of the user's hand or wrist or the user's clothes (sleeve, for example), the processor 24 determines that the first sensor 12 is placed on the chest of the user. If an image of a portion of the user's leg or foot (such as the ankle, instep, calf, and thigh) is captured by the second sensor 14, that is, if the image data indicates a portion of the user's foot or leg, the processor 24 determines that the first sensor 12 is placed on the foot or leg of the user. In this manner, the object (a portion of a user or a portion of the user's clothes) captured by the second sensor 14 varies according to the part where the first sensor 12 is placed. Based on this difference in the object captured by the image capturing operation of the second sensor 14, the processor 24 may identify the part where the first sensor 12 is placed. As the image capturing device, which serves as the second sensor 14, a camera having a wide image-capturing angle (such as a wide angle camera) may be used.

The sixth example may be combined with at least one of the first through fifth examples.

In the above-described first through sixth examples, the first sensor 12 may monitor biological information other than brain waves. That is, in each example, when it is identified that the first sensor 12 is placed at the specific part, the processor 24 may determine that biological information, which may be any type of biological information, monitored by the first sensor 12 is the specific biological information.

The above-described first through sixth examples are only examples. The device 16 may be operated by using biological information other than that discussed in each of the examples. In this case, too, it is judged whether the first sensor 12 is placed at the specific part, based on data measured by the second sensor 14.

In the above-described exemplary embodiment, processing executed by the processor 24 may be executed by a device (an external device, such as a server, for example) other than the information processing apparatus 10. Then, information indicating the processing results may be displayed on the display of the UI 20 or be output as sound information.

In the embodiment above, the term “processor” refers to hardware in a broad sense. Examples of the processor includes general processors (e.g., CPU: Central Processing Unit), dedicated processors (e.g., GPU: Graphics Processing Unit, ASIC: Application Integrated Circuit, FPGA: Field Programmable Gate Array, and programmable logic device). In the embodiment above, the term “processor” is broad enough to encompass one processor or plural processors in collaboration which are located physically apart from each other but may work cooperatively. The order of operations of the processor is not limited to one described in the embodiment above, and may be changed.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus comprising:
a first sensor that monitors specific biological information concerning a user, the specific biological information being used for operating a device;
a second sensor that identifies a part of the user where the first sensor is placed; and
a processor configured to
operate the device in accordance with the specific biological information monitored by the first sensor when the second sensor has determined that the first sensor is placed at a specific part where the specific biological information is monitored.

2. An information processing apparatus comprising:
a first sensor that monitors biological information concerning a user;
a second sensor that identifies a part of the user where the first sensor is placed; and
a processor configured to
operate a device that is assumed to be operated in accordance with specific biological information monitored by the first sensor placed at a specific part, and operate the device in accordance with the specific biological information monitored by the first sensor when the second sensor has determined that the first sensor is placed at the specific part.

3. An information processing apparatus comprising:
a processor configured to
operate a device in accordance with specific biological information concerning a user monitored by a first sensor when a second sensor has determined that the first sensor is placed at a specific part of the user where the specific biological information is monitored, the specific biological information being used for operating the device, the second sensor identifying a part of the user where the first sensor is placed.

4. The information processing apparatus according to claim 1, wherein:
the second sensor is a temperature sensor that measures a temperature of a part where the first sensor is placed; and
if the temperature measured by the temperature sensor is included within a range of temperatures of the specific part, the processor operates the device in accordance with the specific biological information monitored by the first sensor.

5. The information processing apparatus according to claim 2, wherein:
the second sensor is a temperature sensor that measures a temperature of a part where the first sensor is placed; and
if the temperature measured by the temperature sensor is included within a range of temperatures of the specific part, the processor operates the device in accordance with the specific biological information monitored by the first sensor.

6. The information processing apparatus according to claim 3, wherein:
the second sensor is a temperature sensor that measures a temperature of a part where the first sensor is placed; and
if the temperature measured by the temperature sensor is included within a range of temperatures of the specific part, the processor operates the device in accordance with the specific biological information monitored by the first sensor.

7. The information processing apparatus according to claim 4, wherein:
the specific part is an ear or a forehead of the user; and
if the temperature measured by the temperature sensor is included within a range of temperatures of the ear of the user or within a range of temperatures of the forehead of the user, the processor operates the device in accordance with the specific biological information monitored by the first sensor.

8. The information processing apparatus according to claim 1, wherein:
the second sensor is a pressure sensor that measures a pressure level of a part where the first sensor is placed; and
if the pressure level measured by the pressure sensor is included within a range of pressure levels of the specific part, the processor operates the device in accordance with the specific biological information monitored by the first sensor.

9. The information processing apparatus according to claim 2, wherein:
the second sensor is a pressure sensor that measures a pressure level of a part where the first sensor is placed; and
if the pressure level measured by the pressure sensor is included within a range of pressure levels of the specific part, the processor operates the device in accordance with the specific biological information monitored by the first sensor.

10. The information processing apparatus according to claim 8, wherein, if the pressure level measured by the pressure sensor is higher than or equal to a threshold, the processor operates the device in accordance with the specific biological information monitored by the first sensor.

11. The information processing apparatus according to claim 1, wherein:
the second sensor is an illuminance sensor that measures an illuminance level of a part where the first sensor is placed; and
if the illuminance level measured by the illuminance sensor is included within a range of illuminance levels of the specific part, the processor operates the device in accordance with the specific biological information monitored by the first sensor.

12. The information processing apparatus according to claim 2, wherein:
the second sensor is an illuminance sensor that measures an illuminance level of a part where the first sensor is placed; and
if the illuminance level measured by the illuminance sensor is included within a range of illuminance levels of the specific part, the processor operates the device in accordance with the specific biological information monitored by the first sensor.

13. The information processing apparatus according to claim 11, wherein, if the illuminance level measured by the illuminance sensor is lower than or equal to a threshold, the processor operates the device in accordance with the specific biological information monitored by the first sensor.

14. The information processing apparatus according to claim 1, wherein:
the second sensor is a motion sensor that measures an amount of movement of a part where the first sensor is placed; and
if the amount of movement measured by the motion sensor is included within a range of amounts of movement of the specific part, the processor operates the device in accordance with the specific biological information monitored by the first sensor.

15. The information processing apparatus according to claim 14, wherein:
the specific part is a head of the user; and
if the amount of movement measured by the motion sensor is included within a range of amounts of movement of the head of the user, the processor operates the device in accordance with the specific biological information monitored by the first sensor.

16. The information processing apparatus according to claim 1, wherein:
the second sensor is an odor sensor that measures an odor level of a part where the first sensor is placed; and
if the odor level measured by the odor sensor is included within a range of odor levels of the specific part, the processor operates the device in accordance with the specific biological information monitored by the first sensor.

17. The information processing apparatus according to claim 16, wherein, if the odor level measured by the odor sensor is lower than or equal to a threshold, the processor operates the device in accordance with the specific biological information monitored by the first sensor.

18. The information processing apparatus according to claim 1, wherein:
the second sensor is an image capturing device placed at the first sensor; and
if an image capturing range of the image capturing device is a specific range corresponding to the specific part, the processor operates the device in accordance with the specific biological information monitored by the first sensor.

19. The information processing apparatus according to claim 18, wherein the processor identifies a part of the user where the first sensor is placed, based on information indicating a portion of the user or a portion of clothes worn by the user included in an image captured by the second sensor in the image capturing range.

20. A non-transitory computer readable medium storing a program causing a computer to execute a process, the process comprising:
operating a device in accordance with specific biological information concerning a user monitored by a first sensor when a second sensor has determined that the first sensor is placed at a specific part of the user where the specific biological information is monitored, the specific biological information being used for operating the device, the second sensor identifying a part of the user where the first sensor is placed.
